(19) United States
(12) Patent Application Publication
(10) Pub. No.: US 2010/0168231 A1
(43) Pub. Date: Jul. 1, 2010

(54) TARGET ENZYME FOR THE TREATMENT OF ACNE

(75) Inventors: Laurent Lamy, Nice (FR); Michel Rivier, Nice (FR); Gérard Feraille, Mougins Le Haut (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/652,283

(22) Filed: Jan. 5, 2010

Related U.S. Application Data
(63) Continuation of application No. PCT/FR2008/051269, filed on Jul. 7, 2008.

(30) Foreign Application Priority Data
Jul. 6, 2007 (FR) 0756309

(51) Int. Cl. A61K 31/216; A61K 31/11; A61Q 19/00; A61P 17/10; A61P 17/00; A61K 8/37; A61K 8/33; C12Q 1/32; C12Q 1/68; C12N 9/02

(52) U.S. Cl. 514/510; 514/703; 435/26; 435/6; 435/189

(57) ABSTRACT

The use of an enzyme as a research tool for acne, by identifying a compound for the treatment of acne and/or any disorder associated with cutaneous hyperseborrhoea.

### CROSS-REFERENCE TO PRIORITY/PCT APPLICATIONS

This application claims priority under 35 U.S.C. §119 of FR 0756309, filed Jul. 6, 2007, and is a continuation/national phase of PCT/FR 2008/051269, filed Jul. 7, 2008 and designating the United States (published in the French language on Jan. 22, 2009 as WO 2009/010687 A2; the title and abstract were also published in English), each hereby expressly incorporated by reference in its entirety and each assigned to the assignee hereof.

### BACKGROUND OF THE INVENTION

1. Technical Field of the Invention

The present invention relates to the use of an enzyme with dehydrogenase-reductase activity as a research tool in disorders associated with hyperseborrhoea, in particular acne.

The invention also relates to the use of this enzyme in identifying a compound for the treatment of acne, seborrhoeic dermatitis and/or cutaneous disorders associated with hyperseborrhoea, as well as to a method for identifying such a compound.

2. Description of Background and/or Related and/or Prior Art

Acne is a multi-factorial disease characterized by an abnormal production of sebum and ductal cornification followed by bacterial colonization and inflammation. Currently, the most effective product for treating this disease is a synthetic retinoid, 13-cis retinoic acid or isotretinoin (Roaccutane™). However, that product suffers from severe side effects, including teratogenicity.

Although 13-cis retinoic acid is effective against acne when taken orally (“Diagnosis and treatment of acne”, Feldman et al, Am Fam Physician, 2004 May 1; 69(9): 2123-30), its mechanism of action is still poorly understood. The principal advantage of 13-cis retinoic acid is its capacity to significantly reduce the production of sebum (“Effect of oral 13-cis retinoic acid at three dose levels on sustainable rates of sebum secretion and on acne”, Stewart M E et al, J Am Acad Dermatol, 1983 April; 8(4): 532-8 and “Isotretinoin—an explanation for its long-term benefit”, Cunliffe W J et al, Dermatologica, 1987; 175 Suppl 1: 133-7). On the molecular level, 13-cis retinoic acid is much more effective when taken orally than other natural or synthetic retinoids (Cunliffe W J, Norris J F “Isotretinoin—an explanation for its long-term benefit”, Dermatologica, 1987; 175 Suppl 1: 133-7). Original pharmacokinetic properties have been proposed to explain this efficacy.

Certain authors have suggested that 13-cis retinoic acid, which acts on RARs (Retinoic Acid Receptors), could also reduce the production of a metabolite derived from dihydroxytestosterone, 3α-androstanediol glucuronide (“Effect of oral isotretinoin treatment on skin androgen receptor levels in male acneic patients”, Boudou P et al, J Clin Endocrinol Metab, 1995 April; 80(4): 1158-61 and “Isotretinoin, tetracycline and circulating hormones in acne”, Palatsi R et al, Acta Derm Venerol, 1997 September; 77(5): 394-6). Several studies have suggested that the production of sebum is under androgenic control, and an abnormal response of the pilosebaceous unit to androgens seems to be involved in the pathogenesis of acne. These observations are in agreement with the absence of sebum in patients who are entirely insensitive to androgens, and the normal production of sebum in pseudohermaphrodite males. Since sebaceous glands express a series of steroidogenic enzymes, it has been assumed that local transformation of androgenic precursors affects the function of the sebaceous glands, rather than the systemic concentration of DHEA, testosterone or dihydroxytestosterone (DHT).

Recently, certain studies have suggested that Roaccutane could act by modifying intracellular androgenic concentration by inhibiting enzymes involved in the catabolism of the most effective androgen, DHT (“Expression cloning and characterization of oxidative 17beta-and 3alpha-hydroxysteroid dehydrogenases from rat and human prostate”, Biswas M G et al, J Biol Chem, 1997 Jun. 20; 272(25): 15959-66 and “Cloning of the human RoDH-related short chain dehydrogenase gene and analysis of its structure”, Kedishvili N.Y. et al, Chem Biol Interact, 2001 Jan. 30; 130-132(1-3): 457-67).

Currently, at least 6 members of a family of enzymes belonging to the short-chain dehydrogenase-reductase (SDR) superfamily, termed retinol dehydrogenases (RoDHs), have been identified in man. These enzymes are cytosolic or microsomal, function with NAD or NADP as co-factor and are sensitive to different retinoids. Their mechanism of action is multifunctional, since certain of them may have retinol dehydrogenase activity, but also 3α-hydroxy or 17β-hydroxy steroid dehydrogenase activity.

In man, these enzymes include RODH or HSE (RoDH-like 3α-HSD or 3-hydroxysteroid epimerase) (“Expression cloning and characterization of oxidative 17beta- and 3alpha-hydroxysteroid dehydrogenases from rat and human prostate”, Biswas M G et al, J Biol Chem, 1997 Jun. 20; 272(25): 15959-66 and “Cloning of the human RoDH-related short chain dehydrogenase gene and analysis of its structure”, Kedishvili N.Y. et al, Chem Biol Interact, 2001 Jan. 30; 130-132(1-3): 457-67), RoDH-4 or Microsomal NAD+-dependent retinol dehydrogenase 4 (“cDNA cloning and characterization of a new human microsomal NAD+-dependent dehydrogenase that oxidizes all-trans-retinol and 3alpha-hydroxysteroids”, Gough W H et al, J Biol Chem, 1998 Jul. 31; 273(31): 19778-85; “Cloning and characterization of retinol dehydrogenase transcripts expressed in human epidermal keratinocytes”, Jurukovski V et al, Mol Genet Metab, 1999 May; 67(1): 62-73; “Expression pattern and biochemical characteristics of a major epidermal retinol dehydrogenase”, Markova N G et al, Mol Genet Metab, 2003 February; 78(2): 119-35), RoDH5 (Retinol dehydrogenase 5 (11-cis and 9-cis)) (Chetyrkin S V, Hu J, Gough W H, Dumaual N, Kedishvili N Y, “Further characterization of human microsomal 3alpha-hydroxysteroid dehydrogenase”, Arch Biochem Biophys. 2001 Feb. 1; 386(1): 1-10), RDH-E2 (retinal short-chain dehydrogenase) (Expression pattern and biochemical characteristics of a major epidermal retinol dehydrogenase Nedialka G. Markova, et al. Molecular Genetics and Metabolism 78 (2003) 119-135), RoDH11 (dehydrogenase 11 (all-trans and 9-cis)) (“Prostate short-chain dehydrogenase reductase 1 (PSDR1): a new member of the short-chain steroid dehydrogenase/reductase family highly expressed in normal and neoplasic prostate epithelium”, Lin B et al, Cancer Res, 2001 Feb. 15; 61(4): 1611-8; “Evidence that the human gene for prostate short-chain dehydrogenase/reductase (PSDR1) encodes a novel retinal reductase (ralR1)”, Kedishvili N Y et al, J Biol Chem., 2002 Aug. 9; 277(32): 28909-15) and DHRS9 (dehydrogenase/reductase (SDR family) member 9) (“Characterization of a novel type of human microsomal 3alpha-hydroxysteroid dehydrogenase: unique tissue distribution and catalytic properties”, Chetyrkin S V et al, J Biol Chem, 2001 Jun. 22; 276(25): 22278-86; “Characterization of a novel airway epithelial cell-specific short chain alcohol dehydrogenase/reductase gene whose expression is up-regulated by retinoids and is involved in the metabolism of retinol”, Soref C M et al, J Biol Chem, 2001 Jun. 29; 276(26): 24194-202). In contrast to other members of the family, DHRS9 does not have retinol dehydrogenase activity (Chetyrkin S V et al, J Biol Chem, 2001 Jun. 22; 276(25): 22278-86); it has no action on retinoids, and more particularly on retinoic acid.

DHRS9, termed RODH16 in the mouse, has been identified as being present in hair follicles in the same titre as RAR (αβγ) receptors and ALDH1s, but no mention is made as to its level of expression and its major involvement and its role in certain skin pathologies such as acne (Everts et al, Journal of Investigative Dermatology (2007), 127, 1593-1604). It is principally present in the anagenic phases of the hair growth cycle. Furthermore, concerning its presence in mice, since inter-species differences can be very large, it is impossible to extrapolate the presence and level of expression of an enzyme to man.

RoDH-4 and DHRS9 are the only enzymes which have been identified in man which play a role in the formation of DHT and of androstanedione. RODH4 has been described in the publication by T Karlsson et al (Biochem Biophys Res Commun, 2003 Mar. 28; 303(1): 273-278), as an enzyme involved in the stimulation of the secretion of sebum in vitro by the transformation of 3-alphadihydrotestosterone and androsterone into dihydrotestosterone (DHT) and androstanedione. 13-cis retinoic acid (isotretinoin), which is well known for its anti-acne activity, was assumed to reduce the formation of DHT and androstandione in vitro by inhibiting the enzyme RODH4.

### SUMMARY OF THE INVENTION

It has now been discovered that RoDH4 is not expressed (mRNA) in human sebaceous glands and weakly expressed in the epidermis. This weak presence of mRNA was confirmed by using conventional RT-PCR on human epidermis biopsies. The two other sub-types RODH and RODH5 are undetectable in these two skin compartments. RDH11 is strongly expressed in many different tissues with a higher proportion in the spinal cord, prostate, foetal brain, liver, kidney and testicle. In contrast to RDH11, DHRS9 is found in a more restricted number of organs (testicle, heart, marrow and colon) with strong expression in the trachea.

It has now also been discovered that DHRS9 (dehydrogenase reductase member 9) is expressed in human skin, and more particularly in sebocytes and sebaceous glands. This observation is of interest since it means that selective activation or inactivation of this enzyme can be accurately envisaged in the sebaceous glands, in contrast to other sub-types of RoDH (including RODH4) expressed in man. It is now proposed to target the DHRS9 protein in order to prevent and/or treat acne, seborrhoeic dermatitis or any other cutaneous disorder associated with hyperseborrhoea.

The term “acne” means any form of acne, namely and in particular acne vulgaris, comedonal acne, polymorphic acne, nodulocystic acne, acne conglobata, or secondary acnes such as solar acne, acne medicamentosa or occupational acne.

The term “cutaneous disorder associated with hyperseborrhoea” in particular means oily and/or shiny appearance of the skin, the presence of comedones, seborrhoeic dermatitis, and the formation of dandruff.

DHRS9 is expressed in various human cutaneous tissue compartments, but in much larger quantities in the sebaceous glands than in the epidermis. Unless otherwise indicated, the term “DHRS9” means human DHRS9, the sequence for which is recorded in Genbank (NCBI) with accession number NM_{—}005771.

This enzyme could act as an oxidase and convert 3α-androstanediol into DHT, the most effective androgen, in vitro. In contrast to other RoDHs, DHRS9 has no retinoid activity but is regulated by them (“Characterization of a novel airway epithelial cell-specific short chain alcohol dehydrogenase/reductase gene whose expression is up-regulated by retinoids and is involved in the metabolism of retinol”, Soref C M et al, J Biol Chem, 2001 Jun. 29; 276(26): 24194-202).

Modulators of such an enzyme, and more particularly inhibitors, is considered useful in preventing and/or treating acne, seborrhoeic dermatitis or any cutaneous disorder associated with hyperseborrhoea. The term “DHRS9 inhibitor” as used in the context of the invention means any substance, simple or complex compound, of natural or synthetic origin which is capable of inhibiting or reducing the activity or expression of the enzyme DHRS9, the expression of its gene or the activity of at least one of its promoters, and/or which is capable of inhibiting, reducing or slowing down the reaction catalyzed by this enzyme. The inhibitor eliminates or substantially reduces the enzymatic activity of DHRS9. The term “substantially” means a reduction of at least 25%, preferably at least 35%, more preferably at least 50% and still more preferably at least 70% or 90%. More particularly, it may be a compound which interacts with and blocks the catalytic site for the enzyme, like competitive inhibitor type compounds.

Examples of DHRS9 inhibitors are citral and carbenoxolone.

Citral (or lemonal) is the name given to two isomers with empirical formula C₁₀H₁₆O. The two components are diastereoisomers: the trans isomer is known as geranial or citral A. The cis isomer is known as neral or citral B.

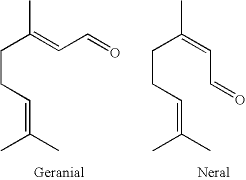

Citral is the major constituent of oil of citronella and of other plants of the genus Cymbopogon. It is also present in verbena, orange and lemon oils.

Carbenoxolone is a synthetic derivative of glycyrrhizinic acid with the formula given below:

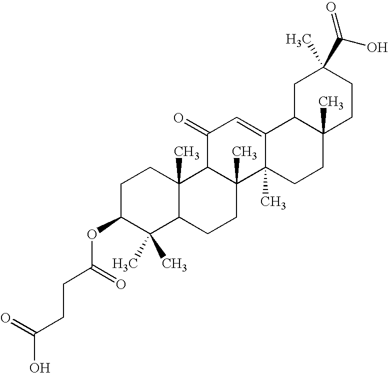

The present invention thus features a method for the preventative and/or curative treatment of acne, seborrhoeic dermatitis or any cutaneous disorder associated with hyperseborrhoea, the method comprising administering a therapeutically effective quantity of a modulator, preferably an inhibitor of the human enzyme DHRS9, to a patient requiring such treatment.

The present invention also features the formulation of a DHRS9 modulator into medicaments for the preventative and/or curative treatment of acne, seborrhoeic dermatitis or cutaneous disorders associated with hyperseborrhoea.

Preferably, the medicaments in accordance with the invention are for the preventative and/or curative treatment of acne or seborrhoeic dermatitis, whether regime or regimen.

Finally, the present invention features the cosmetic application of a modulator for the human enzyme DHRS9 for the aesthetic treatment of oily skin.

### DETAILED DESCRIPTION OF BEST MODE AND SPECIFIC/PREFERRED EMBODIMENTS OF THE INVENTION

Such a modulator, preferably an inhibitor, of the enzyme DHRS9, is useful for formulation into a medicament for the prevention and/or treatment of acne, seborrhoeic dermatitis or cutaneous disorders associated with hyperseborrhoea. Preferably, the medicament is effective in the treatment of acne or seborrhoeic dermatitis. This medicament may be administered orally, parenterally or topically. Preferably, said medicament is for topical application. The term “topical application” means application to the skin or mucous membranes.

The term “orally” means that the pharmaceutical composition may be in the form of tablets, gel capsules, dragées, syrups, suspensions, solutions, powders, granules, emulsions, suspensions of microspheres or nanospheres or of lipid or polymeric vesicles allowing controlled release.

The term “parenterally” means that the pharmaceutical composition may be in the form of solutions or suspensions for perfusion or for injection.

The term “topically” means that the pharmaceutical composition is more particularly for the treatment of the skin and mucous membranes and may be in the form of unguents, creams, milks, pomades, powders, impregnated pads, solutions, gels, sprays, lotions or suspensions. It may also be in the form of suspensions of microspheres or nanospheres or of lipid or polymeric vesicles or polymer patches or hydrogels allowing controlled release. This composition for topical application may be in the anhydrous form, in the aqueous form or in the form of an emulsion.

The composition may include a quantity of enzyme DHRS9 modulator of from 0.001% to 10% by weight, in particular 0.01% to 5% by weight with respect to the total composition weight.

The subject pharmaceutical compositions may also contain inert additives or combinations of said additives, such as:

wetting agents;

flavor improvers;

preservatives such as para-hydroxybenzoic acid esters;

stabilizing agents;

moisture-regulating agents;

pH-regulating agents;

osmotic-pressure modifiers;

emulsifying agents;

UV-A and UV-B filters; and

antioxidants such as alpha-tocopherol, butylhydroxyanisole or butylhydroxytoluene, super oxide dismutase, ubiquinol or certain metal chelating agents.

The present invention also features the use of human enzyme DHRS9 as a research tool for acne, seborrhoeic dermatitis and/or any cutaneous disorder associated with hyperseborrhoea. This enzyme is a novel identification tool, selection tool or tool for characterizing a compound for the treatment of acne, seborrhoeic dermatitis and/or any cutaneous disorder associated with hyperseborrhoea.

This invention also features a method for diagnosing acne or a predisposition to acne, comprising a step for measuring the activity of the enzyme DHRS9 in a biological sample, in particular by measuring the quantity of DHT in said biological sample. This invention also features a diagnostic kit for acne, comprising the enzyme DHRS9.

In accordance with the present invention, a biological sample corresponds to any sample or specimen taken from a living organism, preferably a mammal, in particular a human organism, in a quantity sufficient to be characterized. By way of non-limiting example, the biological sample may be a sample of skin, scalp or mucous membrane, or a cell sample.

In particular, the present invention features the use of human enzyme DHRS9 for identification, selection or characterization of a compound for the treatment and/or prevention of acne, seborrhoeic dermatitis and/or any cutaneous disorder associated with hyperseborrhoea. More particularly, the present invention features the use of the human enzyme DHRS9 to identify a compound useful to prevent and/or improve the symptoms of acne, seborrhoeic dermatitis and/or any cutaneous disorder associated with hyperseborrhoea.

Preferably, the enzyme DHRS9 is employed for the prevention and/or treatment of acne.

This invention also features an in vitro method for screening candidate compounds for the preventative and/or curative treatment of a pathology selected from acne, seborrhoeic dermatitis and cutaneous disorders associated with hyperseborrhoea, comprising determining the capacity of a compound to inhibit the expression or the activity of the human enzyme DHRS9 or the expression of its gene or the activity of at least one of the promoters thereof.

Preferably, the in vitro screening method described above comprises the following steps:

a) preparing at least two biological samples;

b) bringing one of the samples into contact with one or more compounds to be tested;

c) measuring the expression or the activity of the enzyme DHRS9, the expression of its gene or the activity of at least one of the promoters thereof in the biological samples;

d) selecting said compounds for which inhibition of the expression or the activity of the enzyme DHRS9, or inhibition of the expression of its gene or inhibition of the activity of at least one of the promoters thereof in the sample treated in step b) is measured, compared with an untreated sample.

In accordance with a first embodiment, the biological samples are cells transfected with a reporter gene which is operatively linked to all or part of the promoter for the gene coding for the enzyme DHRS9, and step c) described above entails measuring the expression of said reporter gene.

In accordance with a second embodiment, the biological samples are cells expressing the gene coding for the enzyme DHRS9, and step c) described above entails measuring the expression of said gene.

The cell employed may be of any type. It may be a cell expressing the gene for the enzyme DHRS9 in an endogenous manner.

In these methods, expression of the gene for the enzyme DHRS9 or the reporter gene may be determined by evaluating the degree of transcription of said gene or its degree of translation.

The term “degree of transcription” of a gene means the quantity of corresponding mRNA produced. The term “degree of translation” of a gene means the quantity of protein produced.

This invention also features an in vitro method for diagnosis of or monitoring the evolution of acne, seborrhoeic dermatitis or a cutaneous disorder associated with hyperseborrhoea in a subject, comprising comparing the expression or the activity of the enzyme DHRS9, the expression of its gene or the activity of at least one of the promoters thereof, in a biological sample from a subject, with a biological sample from a control subject.

Finally, this invention features an in vitro method for determining the susceptibility of a subject to developing acne, seborrhoeic dermatitis or a cutaneous disorder associated with hyperseborrhoea, comprising comparing the expression or the activity of the enzyme DHRS9, the expression of its gene or the activity of at least one of the promoters thereof, in a biological sample from a subject, with a biological sample from a control subject.

Throughout the present text, unless otherwise specified, the term “expression of the enzyme DHRS9” means the quantity of that enzyme.

The term “activity of the enzyme DHRS9” means its biological activity.

The term “activity of a promoter” means the capacity of that promoter to trigger transcription of the DNA sequence encoded downstream of that promoter (and thus indirectly synthesis of the enzyme DHRS9).

The activity of the enzyme DHRS9 may be evaluated by measuring the quantity of DHT produced, or by measuring the quantity of substrate, 3α-diol, which must disappear, in the samples.

In another embodiment, the present invention features a research tool comprising human enzyme DHRS9.

The research tool could also comprise an analysis of the effects of certain treatments on the activity of DHRS9.

As one example, this research tool could be such that the activity of the enzyme DHRS9 is measured by the quantity of DHT produced.

Finally, this invention features the cosmetic use of a modulator, preferably an inhibitor of the human enzyme DHRS9, for the aesthetic treatment of oily skin.

### BRIEF DESCRIPTION OF THE DRAWINGS

It has now been demonstrated that the human enzyme DHRS9 is expressed in various tissues other than the skin, such as the colon, the bone marrow or the trachea, as shown in Example 1 and FIG. 1. However, DHRS9 is expressed to a very small extent in tissues other than the skin, which is an important advantage, meaning that known systemic side effects linked to modifications in androgen metabolism can be avoided. This low level of expression is the opposite to another possible target: RoDH11, which is strongly expressed in many organs and which is ubiquitous.

In the cutaneous tissue of healthy patients, the enzyme is expressed mainly in the sebaceous glands rather than the epidermis, as can be seen in Example 2 and FIG. 2.

FIG. 3 shows an overall scheme of the pathogenesis of acne, which includes the action of DHRS9.

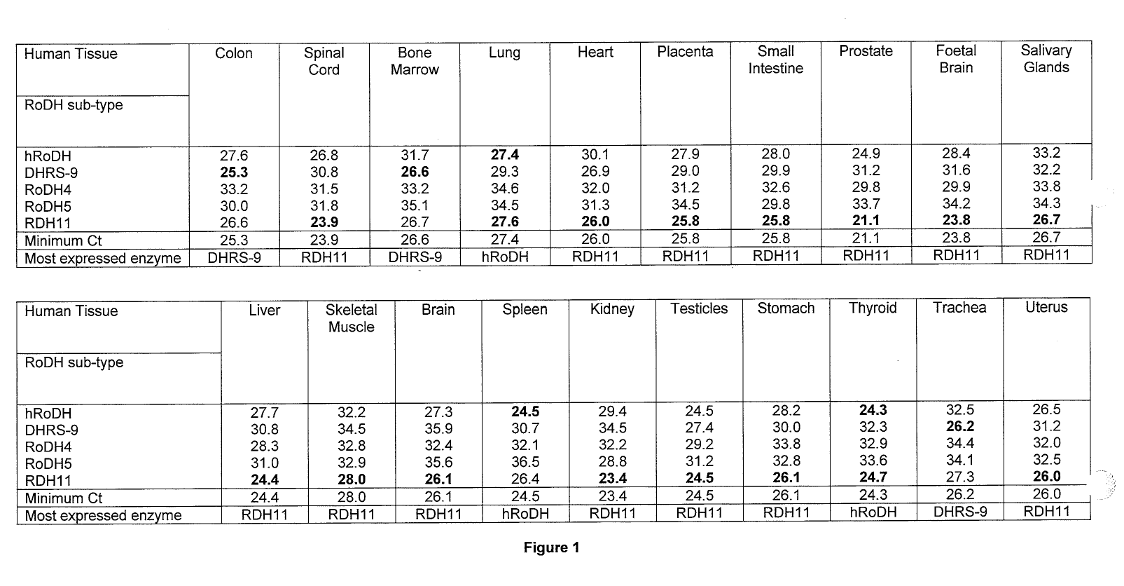

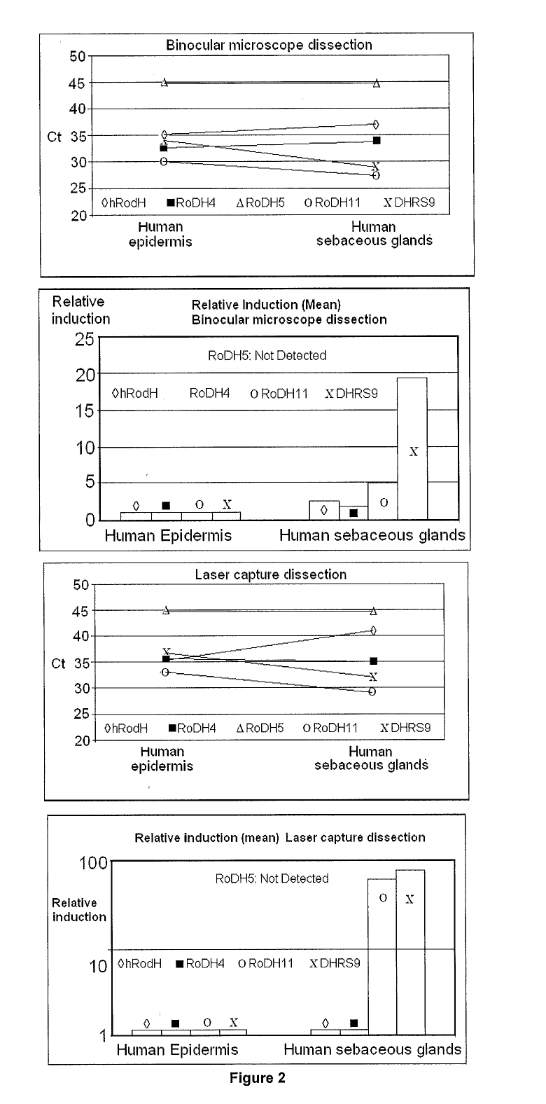

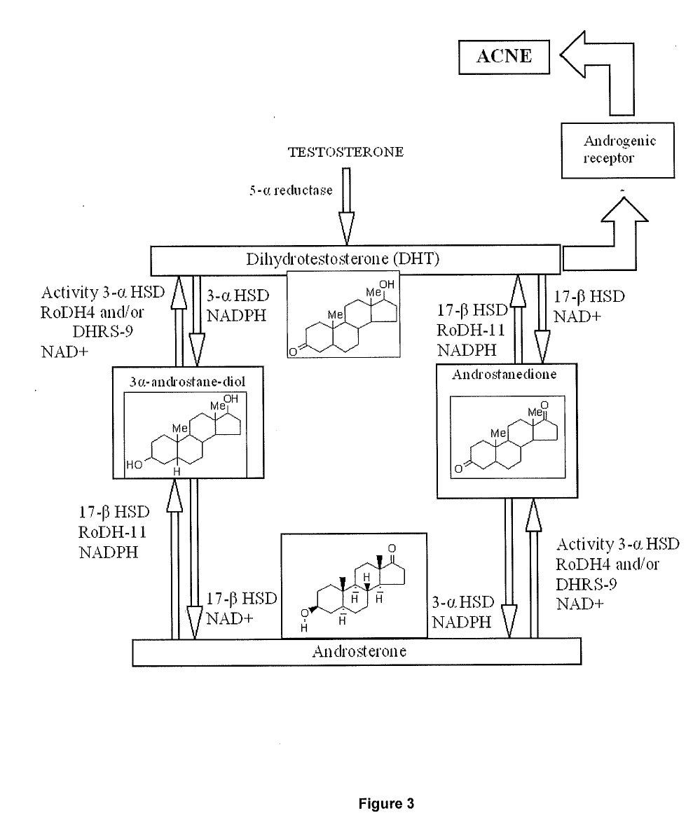

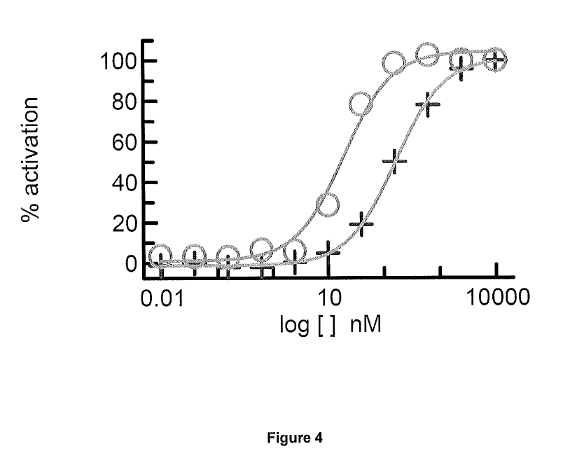

Finally, FIG. 4 shows the conversion activity of 3α-diol to DHT by DHRS9 in cells which are transfected or not transfected with DHRS9.

The observations described above also indicate that the enzyme DHRS9 is a potential therapeutic target for the development of novel therapies for treating acne, seborrhoeic dermatitis or any cutaneous disorder associated with hyperseborrhoea.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Measurement of the Expression of the mRNA Coding for DHRS9 in Various Tissues by PCR

The measurement was carried out by real-time PCR.

Expression of the target genes: hRoDH, RoDH11, RoDH-4, RoDH-5 and DHRS9 was carried out on various human tissues using 5 specific primers.

The results obtained are shown in FIG. 1; they are expressed as the number of cycles necessary to obtain a given fluorescence (Ct: PCR cycle).

The higher the Ct number, the less the target is present in the corresponding tissue.

They show that of the 5 enzymes selected, DHRS9 is primarily expressed in the colon, the spinal cord and the trachea.

### Example 2

### Measurement of the Expression of the mRNA Coding for DHRS9 in the Sebaceous Glands and the Epidermis

This experiment was carried out using samples deriving from two different patients. Dissections of a frozen tissue section were carried out under a binocular microscope and/or by laser microdissection in order to isolate the epidermis and the sebaceous glands. The quantity of mRNA coding for the various enzymes, including DHRS9, was then measured in each of these compartments.

The results are shown in FIG. 2; they are expressed either as the number of cycles necessary to reach a threshold level of fluorescence identical for all of the samples (Ct), or as the relative induction.

They show that DHRS9 is expressed at least 20 times more in the sebaceous glands than in the epidermis alone.

### Example 3

### Cellular Model of Study of Oxidation of 3α-diol by the Enzyme DHRS9

The cDNA coding for human DHRS9 was sub-cloned into a pcDNA3.1/zeo expression vector. This was then transfected into a cell line denoted PALM, standing for PC-3 Androgen receptor Luciferase MMTV. This cell line originates from PC-3 cells which have been stably co-transfected by the expression vector coding for the human androgen receptor (pSG5-puro-h androgen receptor (AR)) and by the reporter gene vector coding for luciferase (pMMTV-neo-Luc), the promoter for which has AR response elements.

The activity of conversion of 3α-diol to DHT by DHRS9 can thus be followed via AR transactivation. The dose-response for the 3α-diol substrate produced an AC₅₀ for AR of 189 nM for non-transfected cells, compared with 19 nM for cells transfected with DHRS9 (see FIG. 4, O: DHRS9 transfected cells, +: non-transfected cells).

By using a reporter gene system, we have shown that DHRS9 is capable of displacing the AC₅₀ of 3α-diol towards the left by approximately one logarithmic order of magnitude, thereby authorizing evaluation of inhibitors (see FIG. 4, O: DHRS9 transfected cells, +: non-transfected cells).

Under the test conditions, 86% inhibition of 3α-diol conversion was attained in the presence of citral in a concentration of 100 μM and 35% inhibition for carbenoxolone at the same concentration.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. An in vitro method for screening and identifying candidate compounds for the preventative and/or curative treatment of a pathology selected from among acne, seborrhoeic dermatitis and cutaneous disorders associated with hyperseborrhoea, comprising determining the capacity of a compound to inhibit the expression or the activity of the human enzyme DHRS9 or the expression of its gene or the activity of at least one of its promoters.

2. An in vitro method for screening and identifying candidate compounds for the preventative and/or curative treatment of acne, seborrhoeic dermatitis or cutaneous disorders associated with hyperseborrhoea as defined by claim 1, comprising the following steps:
a) preparing at least two biological samples;
b) bringing one of the samples into contact with one or more compounds to be tested;
c) measuring the expression or the activity of the enzyme DHRS9, the expression of its gene or the activity of at least one of the promoters thereof in the biological samples;
d) selecting said compounds for which inhibition of the expression or the activity of the enzyme DHRS9, or inhibition of the expression of its gene or inhibition of the activity of at least one of the promoters thereof in the sample treated in step b) is measured, compared with an untreated sample.

3. A pharmaceutical composition comprising an inhibitor of the enzyme DHRS9 in an amount useful for the prevention and/or treatment of acne, seborrhoeic dermatitis or cutaneous disorders associated with hyperseborrhoea.

4. The pharmaceutical composition as defined by claim 3, wherein said inhibitor is selected from among geranial, neral and carbenoxolone.

5. A regime or regimen for the treatment of acne or seborrhoeic dermatitis, comprising administering to a subject in need of such treatment, a thus effective amount of the pharmaceutical composition as defined by claim 3.

6. The pharmaceutical composition as defined by claim 3, formulated for topical application.

7. The pharmaceutical composition as defined by claim 3, formulated for oral administration.

8. A cosmetic composition comprising an inhibitor of the human enzyme DHRS9, in an amount useful for the aesthetic treatment of oily skin.

9. An in vitro method for diagnosing or monitoring the evolution of acne, seborrhoeic dermatitis or a cutaneous disorder associated with hyperseborrhoea in a subject, comprising comparing the expression or the activity of the enzyme DHRS9, the expression of its gene or the activity of at least one of the promoters thereof, in a biological sample from a subject, with a biological sample from a control subject.

10. The method as defined by claim 9, in which the activity of the enzyme is determined by assaying dihydroxytestosterone or 3-alpha-diol in said biological sample.

11. A kit for diagnosing acne, comprising the enzyme DHRS9.

12. An in vitro method for determining the susceptibility of a subject to developing acne, seborrhoeic dermatitis or a cutaneous disorder associated with hyperseborrhoea, comprising comparing the expression or the activity of the enzyme DHRS9, the expression of its gene or the activity of at least one of the promoters thereof, in a biological sample from a subject, with a biological sample from a control subject.
